(19) United States
(12) Patent Application Publication
(10) Pub. No.: US 2014/0121646 A1
(43) Pub. Date: May 1, 2014

(54) Nutrient Absorption Barrier And Delivery Method

(71) Applicants: David Lodin, Maple Grove, MN (US); Kemal Schankereli, Stillwater, MN (US); Howard Lederer, Minnetonka, MN (US)

(72) Inventors: David Lodin, Maple Grove, MN (US); Kemal Schankereli, Stillwater, MN (US); Howard Lederer, Minnetonka, MN (US)

(73) Assignee: FABtec Medical, Inc.

(21) Appl. No.: 13/663,338

(22) Filed: Oct. 29, 2012

(51) Int. Cl. A61M 25/10; A61K 31/11; A61K 31/714; A61K 33/00; A61K 31/131

(52) U.S. Cl. 604/514; 424/666; 514/638; 514/52; 514/705; 514/693; 514/703

(57) ABSTRACT

Methods and devices for preventing nutrient absorption in the small intestine wherein a medical device is passed through the esophagus, and positioned in the small intestine to treat the interior mucosal surface of the small intestine, causing a reduced capacity for nutrient absorption in the treated segment. In one embodiment, the device contains an element that isolates the segment to be treated and allows for the delivery and removal of treatment agents and neutralizing agents.

### FIELD OF THE INVENTION

The present invention relates generally to medical apparatus and methods for temporarily disabling a certain section of that organ, such as a stomach, intestine or gastrointestinal tract to reduce nutrient absorption.

### BACKGROUND OF THE INVENTION

In recent years, there has been very rapid increase in the overall obesity of the world's population. Obesity is defined in terms of Body Mass Index, (BMI), which is expressed as weight in Kg divided by height in meters squared, kg/m2. A BMI greater than 25 is considered overweight, greater than 30 obese, and greater than 40 morbidly obese. According to the World Health Organization, the number of obese people has doubled since 1980, to over 500 million.(1) This trend is also prevalent in the US, where obesity rates have increased from 13.4% in 1960 to 34.3% in 2008.(2) Over the same time period, morbid obesity in the US has risen from 0.9% to 6.0%.(2) In addition to the obesity issues are the concomitant health concerns, most notably the 44% of the world's diabetes problem which is directly attributable to obesity.(1)

With the health concerns associated with such a rapid increase in population obesity, surgical techniques were developed to address the issues where, for numerous reasons, diet and exercise failed.

It was traditionally accepted that bariatric surgery causes weight loss by restriction of gastric volume, intestinal mal-absorption, or a combination of the two. Laparoscopic adjustable gastric banding (LAGB) is considered a purely restrictive procedure that involves the placement of an adjustable band around the cardia of the stomach, creating a 15 ml pouch. (3) Laparoscopic sleeve gastrectomy (LSG) is the resection of the fundus all along the greater curvature of the stomach. LSG was once considered a restrictive procedure, but this presumption has recently come under scrutiny. Bilio-pancreatic diversion (BPD) is an example of a procedure that was considered predominantly mal-absorptive. In this operation, the ingested nutrients are diverted from the stomach to the ileum, bypassing a large segment of proximal bowel. Roux-en-Y gastric bypass (RYGB) traditionally combines both mechanisms, partitioning a small pouch from the proximal stomach and diverting the ingested nutrients to the jejunum with a roux-en-Y gastro-jejunostomy. However, recent investigation suggests additional mechanisms of action including hormonal.

Today, RYGB is the procedure of choice for morbidly obese patients. In additional to the subsequent weight loss, the bariatric surgery has a profound effect on Type II diabetes mellitus (T2DM), initially described in 1995 by Pories et al., who reported that there was an overall T2DM resolution after RYGB of 82.9%. A resolution rate of approximately 80% has been demonstrated repeatedly. It is evident that the anti-diabetic effect is not entirely weight loss as there is a consistent observation that the improvement of glucose and insulin levels occurs within days after RYGB, clearly too soon to be due to the weight loss. The ensuing body of literature has generated two leading theories attempting to explain this weight-independent anti-diabetic effect after RYGB. The ‘hindgut’ proposes that rapid delivery of partially digested nutrients to the distal bowel up-regulates the secretion of incretins such as glucagon-like peptide-1 (GLP-1). The result of the increased incretin secretion is an enhanced glucose-dependent insulin secretion, as well as a number of other changes causing improved glucose tolerance. In the second theory, ‘the foregut hypothesis,’ the exclusion of the duodenum results in the inhibition of a ‘putative’ signal that is responsible for insulin resistance (IR) and/or abnormal glycaemic control. In a non-obese diabetic rat model, surgical diversion of the proximal bowel caused rapid improvement of diabetes without reduction of food intake or change in weight.

More recently, the sleeve gastrectomy has increased in popularity. This is a procedure in which 70-80% of the stomach is effectively removed. This results in a “full” sensation for the patient, plus it is suspected that there is a positive effect on the hormones produced, such as ghrelin, having the effect of reducing hunger.

As the patient population has increased, so have the proposed methods of treatment. Drugs for appetite suppression or the increasing of one's metabolism have been broadly advertised in the US market. Device development approaches include mal-absorption, (as the implantable sleeve from GI Dynamics, and one from Gatrix and Valen Tx), food intake restriction, (such as the banding devices from Allegran and Johnson & Johnson), appetite suppression through nerve stimulation and ablation therapies, (refer to companies such as Enteromedics, Leptus Biomedical and Intrapace), and fillers, intended to occupy a portion of the (typically) stomach and create a “full” sensation for the patient, (the BIB system from Allegran is one such example). All these modalities have shown widely disparate results upon implementation. The one certainty in dealing with this issue is the complex interactions of nerves, hormones, enzymes, and it will likely be a very long time before the interactions are completely understood.

The only technologies which approach the results seen with the surgical techniques are those which rely on mal-absorption. This includes the rapid resolution of T2DM. As a result, there has been the development of barriers which block the absorption of nutrients somewhere along the GI tract, and these devices have demonstrated the best clinical outcomes of the group. Still, those devices under investigation have the distinct disadvantages of being prone to migration and losing position (and effectiveness), having very aggressive anchoring which can be quite damaging to the fragile intestinal tissues, have been seen to cause abrasion in the intestinal wall, which left unchecked can lead to rupture and infection, and all have to be given temporary status causing a mandatory second intervention.

The results to date clearly show that devices inducing mal-absorption offers the greatest benefit to the patient, contributing to significant weight loss as well as resolution of T2 diabetes. The invention presented here, recognizes this and deals with present and severe limitations of other proposed approaches.

### SUMMARY OF THE INVENTION

The present invention provides for improved methods and apparatus for trans-orally providing a barrier to nutrient absorption in the gastro-intestinal tract, most specifically the small intestine. Using the invention described, the physician passes the device beyond the pylorus. The physician then uses the described delivery device to treat a pre-determined length of the GI tract. The treatment can be a physiological change to the local tissue, rendering it non-functional or very limited in function as to its absorption capabilities. This change is temporary as the body will heal the affected area over time, returning to full functionality. The benefit above other devices comes from the lack of a required anchoring mechanism, eliminating a major cause of complications and limitations to implant duration. Equally beneficial is the self-correcting aspect, eliminating the need for re-intervention for device retrieval. Secondarily, this approach offers the ability for re-treatment as warranted by the patient's behavioral modifications or lack thereof.

The length of the treated section may be adjusted by device selection or subsequent treatments by the physician, providing an effective barrier to caloric absorption and which is highly directed and individualized based on the patient's specific needs. The end result is a change in the mucosal surface of the GI tract which results in temporary loss of function of the nutrient absorbing qualities of the treated section. This loss of function provides similar mal-absorption as with the surgical RYGB procedure, without the surgical intervention or permanence, or the inserted barrier devices, but without an implant being left behind, and without the subsequent trauma of anchors and retrieval.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a plan view of an embodiment of a device of the invention;

FIG. 2 is a section view of section A-A of FIG. 1;

FIG. 3 depicts the section of the gastro-intestinal tract being treated with one implementation of the present invention shown.

FIG. 4 depicts a section of a GI tract targeted for treatment using an embodiment of a method of the invention;

FIG. 5 is a detail view of portion of FIG. 4; and

FIG. 6 shows view of tissue targeted for treatment by an embodiment of a method of the invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention described herein describes trans-oral methods to treat obesity and metabolic disease by providing a topical treatment to the mucosal layer of a segment of the gastro-intestinal tract (GI tract) that significantly reduces nutrient absorption in the treated area. For purposes of the present invention, GI tract shall include, but not be limited to, the proximal five feet of the small intestine. As previously discussed, greatly reducing the absorption of nutrients, specifically in the upper small intestine, can have a profound effect on weight loss and the elimination in many patients of T2DM.

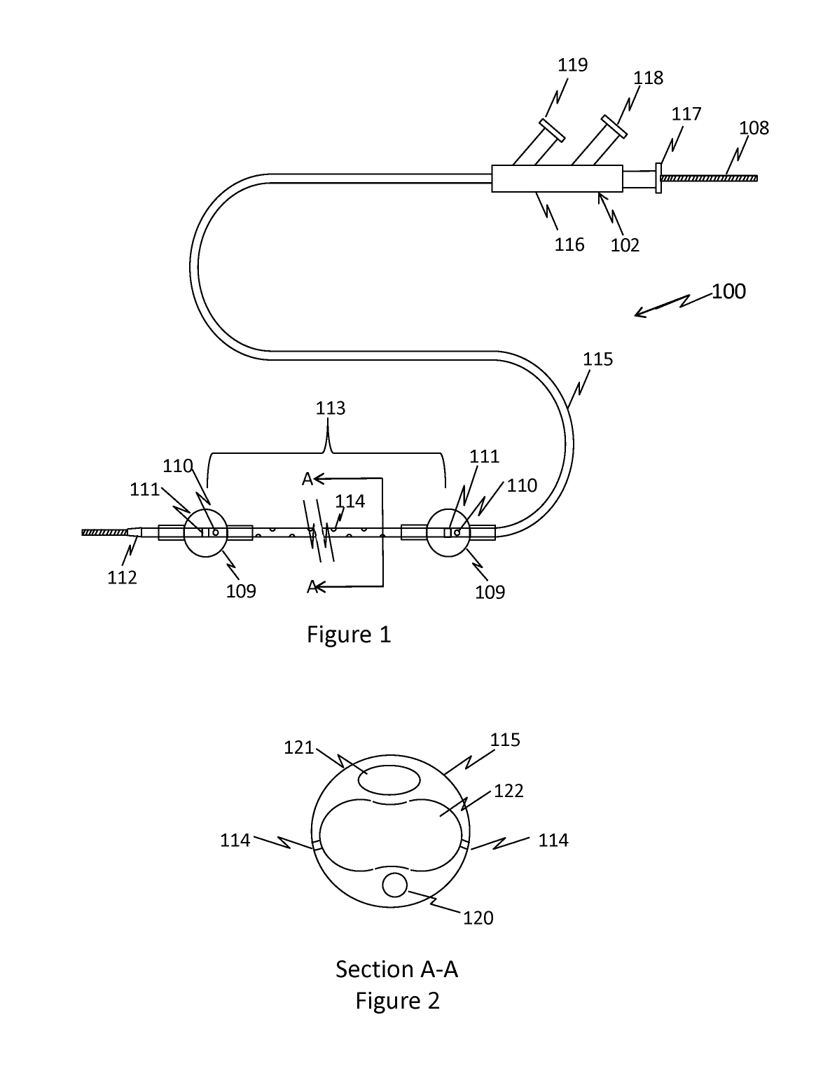

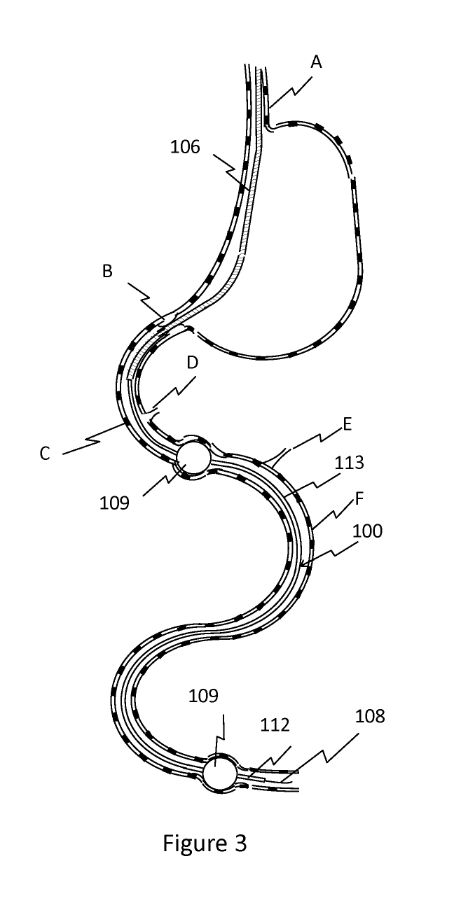

Referring now to FIGS. 1 and 2, there is shown a device 100 of the invention. Device 100 is generally a catheter-style device that is designed for advancement over a guidewire 108 and through a delivery catheter (FIG. 3). Beginning from a proximal end 102, the device 100 includes a manifold 116 defining a guidewire port 117, and a plurality of fluid ports 118 and 119.

Moving along the device 100 in a distal direction, the manifold 102 is connected to a catheter 115, which fluidly connects the manifold 102 with the distal, treatment segment 113 of the device 100. The treatment segment 113 includes a catheter length defined by a plurality of small treatment fluid ports 114, and separated by a pair of balloons 109. In one embodiment, the treatment fluid ports 114 comprise multiple axial rows of ports. The ports of each row may be aligned such that no port is directly across from another port. An inflation port 110 fluidly connects each of the balloons 109 with an inflation lumen 121, internal to the catheter 115, shown in FIG. 2. The inflation lumen 121 is proximally terminates at fluid port 119. Multi-lumen catheter 115 also includes a central lumen 122 that carries treatment fluid from fluid port 118 to the small treatment fluid ports 114. Treatment fluid ports 114 run the entire length of the treatment section 113 and are spaced from 1-10 cm, preferably 2 cm. Preferably, there are two rows of distal ports accessing the lumen 122 diametrically opposed and spaced so that one side of ports is mid-way between the other. Multiple ports will aid to uniformly and rapidly distribute the treatment solution and the alternate spacing will reduce kinking in the device.

One or more radiopaque markers 111 are provided to assist in visualizing one or both ends of the treatment segment 113. At the distal end of the device, a conical tip 112 is provided to create a smooth, atraumatic transition between a guidewire 108 and the device 100. Additionally, a guidewire lumen 120 runs the length of the device 100 between the tip 112 and the guidewire port 117.

Having described the basic components of the device 100, attention is now drawn to FIG. 3 to explain the general method of the invention. FIG. 3 depicts an embodiment of the invention in which the mucosal surface is treated to cause reduced nutrient absorption. Using an imaging modality, such as fluoroscopy, a guidewire 108 and a sheath catheter 106 are passed through the GI tract A across the pylorus B. With the guidewire 108 positioned distal of the target area for treatment, the device 100 is then passed through the catheter 106 along the guide wire 108 and positioned to the proper location, using markers 111, which are optimally located under the balloons 109 or adjacent thereto. The balloons 109 are then inflated to isolate the section of intestine to be treated. Proper placement and treatment area may include the entirety of the duodenum B and jejunum C with preference given to the section distal to the sphincter of oddi D. Further preferred location could locate the treatment area entirely within the jejunum, beginning immediately distal the ligament of trietz E.

To ensure isolation, the balloons must be inflated significantly larger than the diameter of the intestine, up to an inflated diameter of up to 200% of the diameter of the intestine, preferably to 150% of the intestinal diameter. Techniques to produce balloons as are described are well understood. Inflation of the balloons involves infusion of (typically) normal saline through port 119. The inflation fluid may or may not contain a radiopaque dye for observation and confirmation of inflation and deflation.

With a section of the intestine to be treated properly isolated, a solution to affect treatment of the mucosal lining of the small intestine is introduced through fluid port 118, exiting treatment fluid ports 114. After treatment is completed balloons 109 are then deflated by placing suction on inflation port 119. The device 100, catheter 106 and guidewire 108 are then removed and the procedure is complete.

It is to be understood that specific treatment protocols will vary depending on the concentrations of the solutions used, the size and condition of the individual patient, the patient's treatment history, and numerous other variables. As such, the general method just described is further explained more specifically by way of the following examples:

### Example I

An acidic solution is formulated using an organic or inorganic acid. The solution is intended to digest the epithelial elements of the mucosa rendering them ineffective in absorption of nutrients. A second effect of the digestion process will be to disrupt the neural pathways affecting nutrition uptake distal to the affected location. While the pH of the acidic solution may vary from 0.1 to 5, positive results have been achieved using a pH in the range of 0.5 to 1.5. Starting with 100 ml of 2 N HCl solution the pH is modified using NaOH using such that final pH is in the range of 0.5 to 1.5. A volume of the modified solution is drawn into a syringe of appropriate size and fixed onto the proximal port 118 of the isolated intestine and the solution is introduced such that the isolated intestinal mucosa is entirely immersed with the solution. The residence time of the solution in the intestine may be from 30 seconds to 15 minutes. Following, the isolated intestine is aspirated through the same infusion ports, then flushed to remove the acid solution. The first flush of the intestine is conducted using water and is intended only to remove the digesting solution from the intestinal lining. Secondarily, any residual acid is neutralized using a buffer system of adequate strength (molar concentration) and pH to affect neutrality, pH 7.0, of the intestinal environment.

While many different buffering systems can be envisioned one particularly useful system would consist of a buffering solution formulated from mono and dibasic phosphate salts such that the final buffering concentration is in the range of 0.01-3.0 molar concentration with positive results at approximately 0.2 M.

The buffer is introduced into the proximal access port and the mucosal aspect of the intestine is flushed for a period of 30 seconds to 15 minutes. The lack of effervescence of the exiting buffer solution will indicate completion of the rinsing process.

Subsequently the proximal and distal isolating balloons are removed and the modified intestinal tract is allowed to function.

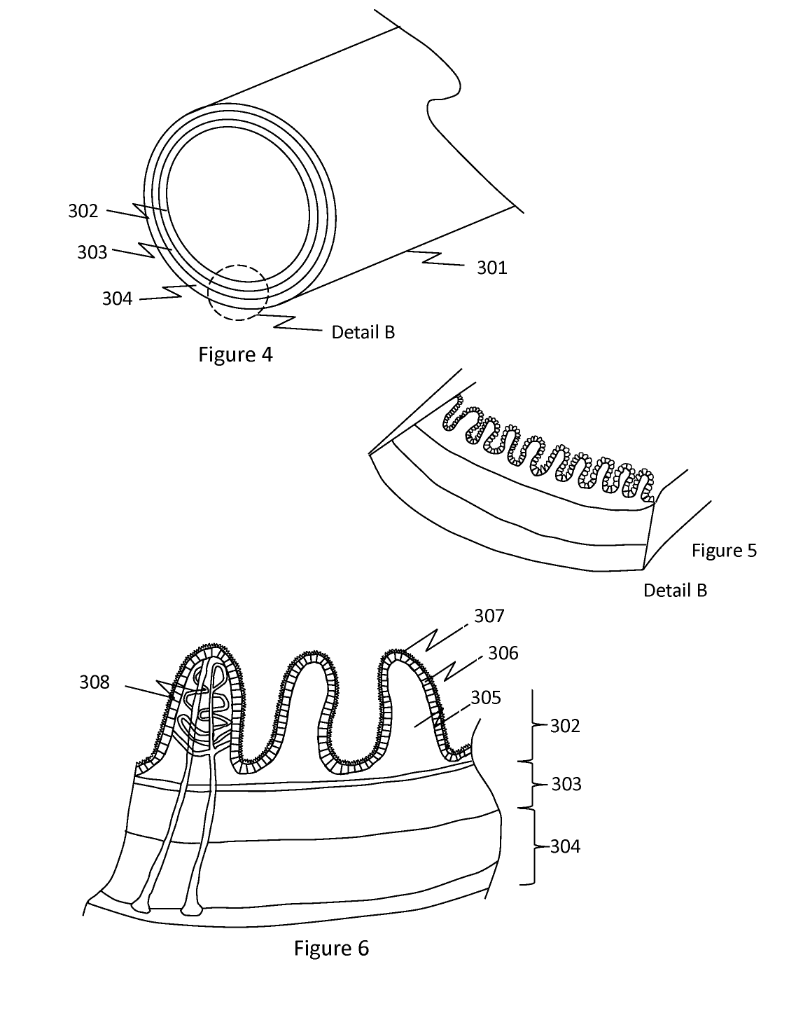

With reference to FIGS. 4-6, the resulting affect on the mucosal tissue is a mild erosion of the mucosal layer 302. This will be evidenced by the removal of part or all of the villi 305 and its components. Further, the erosion may go into the sub-mucosal layer 303. This would be appropriate when a longer lasting effect of treatment, (reduced nutrient absorption) is warranted. Deeper tissue removal results in longer healing times and therefore longer interruption in normal tissue function. It is desired not to significantly treat into the muscular layer 304 so as not to impact the peristaltic function of the intestine, keeping the body's mechanism to move food along the GI tract intact.

### Example II

A fixative solution is formulated such that the proteinaceous components of the mucosa and mucosal epithelium are crosslinked rendering them ineffective in supporting nutrition uptake. While crosslinking can be affected using varying agents such as carbodiimide, vitamin B12/UV, and mono and dialdehydes. One preferred crosslinker is gluteraldehyde.

A crosslinking solution is formulated using gluteraldehyde. Starting from a base solution of 25% gluteraldehyde the solution is diluted using a buffering solution such as 0.02 M phosphate such that the final concentration of 0.1-5% v/v gluteraldehyde with a preferred concentration being 1%. The pH of the solution is secondarily adjusted to 7.0.

A volume of the crosslinker is loaded into a syringe and the solution is delivered into the proximal portion of the isolated intestinal section until the mucosa is fully immersed. The solution is left in contact with the mucosa for a period of 30 seconds to 30 minutes with a preferred time being approximately five (5) minutes.

Following adequate exposure the gluteraldehyde solution is rinsed from the intestinal tract using a buffering solution such as 0.2 M phosphate buffer at pH 7.0. To assure complete removal of the aldehyde functionality from the intestinal tract a secondary rinse is conducted using an aldehyde scavenger. One particularly useful scavenger is the amino acid glycine. Thus an aqueous solution containing 1% glycine v/v is passed through the isolated intestine to assure complete inactivation of any residual aldehyde.

Subsequently the device is removed and the modified intestinal tract is allowed to function.

The resulting affect on the cross-linked mucosal tissue 302 will be to eliminate all or part of the nutrient absorption functionality of the treated section. Left to time, the treated area section is will heal, thereby making the procedure self-reversing. Deeper cross-linking into the sub-mucosal layer 30 would be appropriate when a longer lasting effect of treatment, (reduced nutrient absorption) is warranted. Deeper tissue cross-linking results in longer healing times and therefore longer interruption in normal tissue function. It is desired not to significantly treat into the muscular layer 304 so as not to impact the peristaltic function of the intestine, keeping the body's mechanism to move food along the GI tract intact.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method for treating obesity and metabolic diseases comprising:
changing tissue nutrient absorption characteristics of an internal wall of a segment of small intestine by exposing said internal wall to a treatment agent;
establishing interaction between said agent and said internal wall until said segment is rendered ineffective in nutrient absorption;
terminating said interaction between said agent and said internal wall.

2. The method of claim 1 wherein exposing said internal wall to a treatment agent comprises exposing said internal wall to a cross-linking agent.

3. The method of claim 1 wherein exposing said internal wall to a treatment agent comprises exposing said internal wall to an etching agent, temporarily depleting cells responsible for nutrient absorption.

4. The method of claim 1, wherein terminating said interaction between said agent and said internal wall comprises terminating said interaction between said agent and said internal wall before permanent change is induced.

5. The method of claim 1, wherein terminating said interaction between said agent and said internal wall comprises introducing a buffering agent.

6. The method of claim 1 wherein terminating said interaction between said agent and said internal wall comprises flushing said agent from said segment.

7. The method of claim 2, wherein exposing said internal wall to a cross-linking agent comprises exposing said internal wall to a cross-linking agent selected from the group consisting of carbodiimide, vitamin B12/UV, monoaldehydes, dialdehydes, and gluteraldehyde.

8. The method of claim 3, wherein exposing said internal wall to an etching agent comprises exposing said internal wall to an etching agent selected from the group consisting of acid, organic acid, inorganic acid.

9. The method of claim 1 wherein exposing said internal wall to a treatment agent comprises:
introducing a catheter having a proximal balloon and a distal balloon axially spaced from said proximal balloon a distance approximating a desired length of said segment such that said proximal balloon is located at a desired proximal end of said segment and said distal balloon is located at a desired distal end of said segment;
inflating said proximal and distal balloons with a fluid, thereby isolating said segment;
passing said agent through a lumen of said catheter having exit ports between said proximal and distal balloons such that said agent exits said catheter through said exit ports and contacts said internal wall of said segment of small intestine.

10. The method of claim 8 wherein said etching agent comprises HCl acid.

11. The method of claim 5 wherein said buffering agent has a concentration of 0.01 to 3.0 molar.

12. The method of claim 8 wherein said etching agent comprises a solution having a pH in the range of 0.1 to 5.

13. A method for treating obesity and metabolic diseases comprising treating an internal wall of a small intestine with an etching agent, temporarily depleting cells responsible for nutrient absorption.

14. The method of claim 13, wherein treating an internal wall of a small intestine with an etching agent comprises treating an internal wall of a small intestine with an etching agent selected from the group consisting of acid, organic acid, inorganic acid.

15. The method of claim 13 wherein treating an internal wall of a small intestine with an etching agent comprises isolating a segment of small intestine to be treated using balloons and introducing said etching agent between said balloons with a catheter.

16. The method of claim 13 wherein treating an internal wall of a small intestine with an etching agent comprises controlling effects of said etching agent using a buffering agent.

17. A method of reducing a nutrient absorption characteristic of a segment of a GI tract comprising:
isolating a segment of a GI tract;
introducing a cross-linking agent into said isolated segment; and,
removing said cross-linking agent.

18. The method of claim 17 wherein introducing a cross-linking agent comprises introducing a cross-linking agent selected from the group consisting of carbodiimide, vitamin B12/UV, monoaldehydes, dialdehydes, and gluteraldehyde.

19. The method of claim 17 wherein isolating a segment of a GI tract comprises inflating balloons at ends of said segment.

20. The method of claim 17 wherein introducing a cross-linking agent into said isolated segment comprises using a catheter to deliver a cross-linking agent into said isolated segment.

21. A method of reducing a nutrient absorption characteristic of a segment of a GI tract comprising cross-linking tissue of an internal wall of said segment.
